(19) United States
(12) Patent Application Publication
(10) Pub. No.: US 2008/0193553 A1
(43) Pub. Date: Aug. 14, 2008

(54) Chinese Patent Medicine for Treating Female Mycotic Leucorrhea Gonorrhea

(76) Inventor: Yanqun Jiang, Fu Jian (CN)

Correspondence Address: ERIC CHAN, 42 PIN OAKS DRIVE, PHOENIXVILLE, PA, 19460, US

(21) Appl. No.: 11/995,686

(22) PCT Filed: Nov. 18, 2005
(86) PCT No.: PCT/CN2005/001959

§ 371 Date: Jan. 14, 2008

(30) Foreign Application Priority Data
Aug. 17, 2005 (CN) 200510019324.0

(51) Int. Cl. A61K 35/56; A61K 36/00; A61P 31/10

(52) U.S. Cl. 424/547; 424/777; 424/775

(57) ABSTRACT

This invention makes public a kind of Chinese patent medicine for treating female mycotic leucorrhea, gonorrhea. It includes 8-20 weight portions of veronica, 12-30 weight portions of fruit of Chinese azalea, 30-40 weight portions of greenbrier, 15-20 weight portions of fruit of glossy privet. Fleece-flower root, leonurus, “Cao Xian”, calcined oyster shell, and “Gui Zhong” can also be added to it to increase the curative effect. Cuidium officiuala, Chinese yam, agalloch and tree peony bark can also be added to it the achieve the effect of dispelling wind and promoting blood circulation. Fox-glove and drug of solomonseal can also be added to it to further achieve the effect of tonifying “qi” and improving one's look. Summing up the pesticide effect, this invention is a Chinese patent medicine for treating female mycotic leucorrhea, gonorrhea. It can eliminate mycotic, clear away the toxin in the body, cure effectively itching, red and swollen, metro-erosion, and other diseases infected by leucorrhea, gonorrhea, thus to cue thoroughly mycotic leucorrhea, gonorrhea. Besides, the Chinese herbal medicines adopted by this invention are all commonly-seen medicines and their prices are rather low.

### BACKGROUND OF THE INVENTION

This invention involves a kind of Chinese patent medicine, especially involves a kind of Chinese patent medicine for treating female mycotic leucorrhea, gonorrhea.

Mycotic leucorrhea, gonorrhea are a kind of commonly-seen disease whose disease incidence is rather high. Due to the reason of reproduction structure women are easy hypersensibility, mycetome and they are of weak resistance to disease. They are easily infected before and after menstrual period, thus leucorrhea and gonorrhea are more common. If it is the response to acute, chronic inflammation which does not remove in time and stop its transmission, it may evolve to pathological changes in uterus, metro-erosion, and others. There are quite a lot of medicines, both western medicine and TCM, for treating this disease. It cures quickly, but once you discontinue taking medicine, it will come back quickly. It cures the symptoms, not the disease, it cannot kill the mycotic thoroughly, and cannot eliminate the toxin, and its price is rather high.

### BRIEF SUMMARY OF THE INVENTION

The aim of this invention is to provide a kind of Chinese patent medicine for treating female mycotic leucorrhea, gonorrhea which is of good curative effect and low price.

The technological plan of this invention is like this: a kind of Chinese patent medicine for treating female mycotic leucorrhea, gonorrhea which includes the following ingredients: 8-20 weight portions of veronica, 12-30 weight portions of fruit of Chinese azalea, 30-40 weight portions of greenbrier, and 15-20 weight portions of fruit of glossy privet.

The best portions of above-mentioned veronica, fruit of Chinese azalea, green brier, and fruit of glossy privet are 10 weight portions, 20 weight portions, 30 weight portions, and 20 weight portions respectively.

It also includes the following ingredients: 10-25 weight portions of fleece-flower root, 15-30 weight portions of leonurus, 10-20 weight portions of “Cao Xian” (a kind of moss), 15-35 weight portions of calcined oyster shell, and 8-15 weight portions of “Gui Zhong” (a kind of Chinese herbal medicine).

The best portions of the above-mentioned fleece-flower root, leonuru, “Cao Xian”, calcined oyster shell, and “Gui Zhong” are 15 weight portions, 20 weight portions, 15 weight portions, 25 weight portions, and 10 weight portions respectively.

It also includes the following ingredients: 8-18 weight portion of cuidium officiuala, 10-20 weight portions of Chinese yam, 8-15 weight portions of agalloch, and 10-18 weight portions of tree peony bark.

The best portions of above-mentioned cuidium officiuala, Chinese yam, agalloch, and tree peony bark are 12 weight portions, 15 weight portions, 12 weight portions, and 15 weight portions respectively.

It also includes the following ingredients: 8-20 weight portions of fox-glove, and 18-30 weight portions of drug of solomonseal.

The best portions of the above mentioned fox-glove, and drug of solomonseal are 15 weight portions, and 20 weight portions respectively.

The main prescription of this invention is veronica, fruit of Chinese azalea, greenbrier, and fruit of glossy privet.

Veronica (also known as nine-layer pagoda, a Chinese herbal medicine), is hot and sweet in taste, warm in nature. When entering into lung, spleen, stomach, and large intestine, it has the effects of dispelling wind and removing cold, promoting the flow of “qi” and blood circulation, removing dampness to restore function, and clearing toxic material and reducing swelling. Its main function is to treat catching cold (fearing cold, little and thin sputum, thin and white fur, and others), indigestion, anorexia, ventosity, diarrhea menoxenia, and others. It can relieve lumbago after woman's delivery and help children grow.

The fruit of Chinese azalea is warm in nature, and hot in taste. It has the effect of venomous, expelling wind, and analgesic therapy.

The greenbrier is light sweet in taste, neutral in nature, It can induce diuresis, clean away heat and toxic material. It has prominent curative effect in clinical treatment of malignant sore and ulcer.

The fruit of glossy privet is bitter in taste, cold in nature. It is used generally as tonic, but taking more of it may cause loose bowels. Its drug form of decoction is commonly used to treat TB, hot flash, backache, edema, and others, and its drug form of pill and paste is commonly used to treat dizzy head and blurred eyes, palpitation, and others. Taking by mixing with black sesame, it is commonly used to treat progeria, panasthenia, and others. This invention can increase its curative effect by adding fleece-flower root, leonurus, “Cao Xian”, calcined oyster shell, and “Gui Zhong”.

The fleece-flower root is bitter sweet in taste, tepor in nature, it can tonify kidney and supplement sperm. It is god for kidney function and increases the reproduction ability. It is used to supplement sperm, to treat the weakness of both liver and kidney, and to treat dizzy head and blurred eyes, tinnitus, insomnia, progeria, pain in waist and knee, nocturnal emission all of which are caused by insufficient sperm and blood. It can also be used to treat female leucorrhea after delivery and long-occurring malaria.

The leonurus is slightly cold in nature, bitter and hot in taste. It can promote blood circulation and regulate menstruation, and induce diuresis and subside swelling. It is used to treat irregular menstruation, paramenia, amenorrhea, lochia, edema and oliguria, and acute nephritis edema.

To achieve the effect of dispelling the wind and promoting blood circulation, cuidium officiuala, Chinese yam, agalloch, and tree peony bark can be added to this invention. By adding fox-glove and drug of solomonseal to this invention, it can achieve the effect of tonifying “qi” and improving one's look, and it makes the incretion regulate basically, and it can also achieve the effect of good complexion and of natural health and beauty. Summing up the pesticide effect of above-mentioned Chinese medicinal crops, this invention is a Chinese patent medicine for treating female mycotic leucorrhea, gonorrhea. It can clear away directly the toxin in the privates, clean the uterus inflected by mycotic, it can also expel the poisonous bacteria in abdomen, clear away the toxin in body. It can treat effectively the itch, red and swollen erosion, and other diseases inflected by leucorrhea, gonorrhea. Thus it cures basically the focus. The Chinese herbal medicines adopted are all commonly-seen medicines, their medicinal source are convenient and their prices are reasonable.

### DETAILED DESCRIPTION OF THE INVENTION

This invention is a kind of Chinese patent medicine for treating female mycotic leucorrhea, gonorrhea. It includes the following ingredients: 10 g veronica, 20 g fruit of Chinese azalea, 30 g greenbrier, and 20 g fruit of glossy privet.

For the purpose of increasing curative effect, 15 g fleece-flower root, 20 g leonurus, 15 g “Cao Xian”, 25 g calcined oyster shell, and 10 g “Gui Zhong” can be added to it.

For achieving the effect of dispelling wind and promoting blood circulation, 12 g cuidium officiuala, 15 g Chinese yam, 12 g agalloch and 15 g tree peony bark can be added to it.

For achieving further effect of tonifying “qi” and improving one's look, 15 fox-glove and 20 g drug of solomonseal can be added to it.

The fabricating method is as follows: take out and weigh the above-mentioned Chinese medicinal crops according to their proportion. At first refine veronica, fruit of Chinese azalea, into concentrated liquid for reservation. Then under 60° C. warmly fry and dry greenbrier, fleece-flower root, fruit of glossy privet, leonurus, “Cao Xian”, calcined oyster shell, “Gui Zhong”, cuidium officiuala, Chinese yam, agalloch, tree peony bark, fox-glove, and drug of solomonseal, then grind them into powder, and afterwards, make banburying the powder with the concentrated liquid of veronica and fruit of Chinese azalea into pills.

Dosage: twice a day, 4-6 g each time. Taking the medicine continuously for 10-15 days. The time to take medicine for patient of serious mycotic will be longer until bringing the illness under permanent control.

This invention has experienced clinical trial for several years. The case age from 16 to 55 years old. There are hundred cured cases in the trial.

Case 1: Ms Shi, 33 years old, with pale and severe black eye socket. After examination, she suffered from positive anaphylactic inflection. Her privates has red and swollen, itching inside and outside and burning pain. Her menstruation is irregular and her itching and pain are more serious before and after menstruation. She was in hospital for 10-plus-days treatment before taking this medicine, her illness returned after several days of curing, she re-treats and repeats it again and again, but she fails to cure it. She becomes weaker and weaker. And her resistant ability decreases. Then Ms. Shi took this medicine, twice a day, 6 g each time after meal. From first day to third days, she felt a slight pain in her stomach. On the sixth day, some of the itching and pain were dispelled. The red and swollen in her privates faded away. On the 14^{th} day of taking medicine, all the symptoms disappeared. In addition, she washed her cunus with vinegar water. After 20 days her countenance was normal and her illness was cured. There are 42 cases of the similar case, the age was between 25-42. The rate of cure once and for all reached 90% and more.

Case 2: Ms. Wang, 28 years old, was infected by mycotic and toadstool. She suffered from itch and pain in privates and there produced some fine little clams. She could not take care of herself. She treated in hospital for 4 months, but her illness state was changeable. Her illness was more severe every return. Then she took this medicine three times a day, 6 g each time, and soak externally with vinegar eater. She got effect after 3 days and her body can move about. Itching and pain in her privates were dispelled after 19 days. The experiment result after 25 days proved that all the indexes restored normally, but her body was still weak. She took this medicine for 52 days to consolidate and her illness did not return till now. There are 12 cases of the similar case (of illness), the age is between 24-44 year old. Among them 12 cases had curative effect and 8 cases were cured once and for all.

Case 3: Miss Lin, 19 years old, suffered from leucorrhea and gonorrhea. Her underwear was often wet, her menstruation was abnormal and her countenance was dark and light. She recovered after taking this medicine for 12 days. There are 60-plus cases of the similar case (of illness). The age is between 16-15 years old. The curative rate reached 90%

What is claimed is:

1. A kind of Chinese patent medicine for treating mycotic leucorrhea, gonorrhea; it is featured by the following ingredients: 8-20 weight portions of veronica, 12-30 weight portions of fruit of Chinese azalea, 30-40 weight portions of greenbrier, and 15-20 weight portions of fruit of glossy privet.

2. A kind of Chinese patent medicine for treating female mycotic leucorrhea, gonorrhea as in claim 1, wherein it is featured by the best portions of the said Veronica, fruit of Chinese azalea, greenbrier and fruit of glossy privet which are 10 weight portions, 20 weight portions, 30 weight portions and 20 weight portions respectively.

3. A kind of Chinese patent medicine for treating female mycotic leucorrhea, gonorrhea as in claim 1, wherein it is featured by including the following ingredients: 10-25 weight portions of fleece-flower root, 15-30 weight portions of leonurus, 10-20 weight portions of “Cao Xian”, 15-35 weight portions of calcined oyster shell and 8-15 weight portions of “Gui Zhong”.

4. A kind of Chinese patent medicine for treating female mycotic leucorrhea gonorrhea as in claim 3, wherein it is featured by the best portions of the said fleece-flower root, leonurus, “Cao Xian”, calcined oyster shell and “Gui Zhong” which are 15 weight portions, 20 weight portions, 15 weight portions, 25 weight portions, and 10 weight portions respectively.

5. A kind of Chinese patent medicine for treating female mycotic leucorrhea, gonorrhea as in claim 1, wherein it is feature by including also the following ingredients: 8-18 weight portions of cuidium officiuala, 10-20 weight portions of Chinese yam, 8-15 weight portions of agalloch, and 10-18 weight portions of tree peony bark.

6. A kind of Chinese patent medicine for treating female mycotic leucorrhea, gonorrhea as in claim 5, wherein it is featured by the best portions of the said cuidium officiuala, Chinese yam agalloch and tree peong bark which are 12 weight portions, 15 weight portions, 12 weight portions and 15 weight portions respectively.

7. A kind of Chinese patent medicine for treating female mycotic leucorrhea, gonorrhea as in claim 1, wherein it is featured by including also the following ingredients: 8-20 weight portions of fox glove and 18-30 weight portions of drug of solomonseal.

8. A kind of Chinese patent medicine for treating female mycotic leucorrhea, gonorrhea as in claim 7, wherein it is featured by the best portions of the said fox-glove and drug of solomonseal which are 15 weight portions and 20 weight portions respectively.
